(19) United States
(12) Patent Application Publication
(10) Pub. No.: US 2026/0364324 A1
(43) Pub. Date: Nov. 19, 2026

(54) GASTRIC RESIDENCE ARTICLE

(71) Applicant: Junion Therapeutics (Xiamen) Co.,Ltd., Xiamen (CN)

(72) Inventors: Jingnan LUO, Xiamen (CN); Bohuai Moses LEE, Xiamen (CN); Xiaoyi LIN, Xiamen (CN)

(73) Assignee: Junion Therapeutics (Xiamen) Co.,Ltd., Xiamen (CN)

(21) Appl. No.: 19/734,131

(22) Filed: Jul. 8, 2026

Related U.S. Application Data
(63) Continuation of application No. PCT/CN2025/070895, filed on Jan. 7, 2025.

(30) Foreign Application Priority Data
Jan. 9, 2024 (CN) 202410028344.7

(51) Int. Cl. A61M 31/00

(52) U.S. Cl. CPC A61M 31/002; A61M 2210/1053

(57) ABSTRACT

The present invention relates to the field of gastric residence systems and discloses a gastric residence article comprising an elastic central component, a plurality of deployment arms, and a plurality of elastic end caps. The plurality of deployment arms are circumferentially fixed around the elastic central component and extend radially therefrom. The elastic end caps are disposed at the free ends of the deployment arm. The elastic end caps are configured to undergo at least 20% elastic deformation in the direction of pressure when subjected to a pressure of 2N. The provision of the elastic end caps effectively prevents scratching of the gastrointestinal inner wall tissue.

### CROSS-REFERENCE TO RELATED APPLICATIONS

The application claims priority to Chinese patent application No. 202410028344.7, filed on Jan. 9, 2024, the entire contents of which are incorporated herein by reference.

### TECHNICAL FIELD

The present invention relates to the field of gastric residence systems, and in particular, to a gastric residence article.

### BACKGROUND

Gastric residence articles are designed for administration into a patient's stomach, typically swallowed or introduced via other delivery methods (e.g., feeding tubes or gastric tubes) in capsule form. Once the capsule dissolves in the stomach, the gastric residence article expands or deploys to a certain size that retains it within the stomach and resists passage through the pyloric sphincter for a desired residence period (e.g., three days, seven days, two weeks, etc.). This requires maintaining mechanical stability during the desired residence period while releasing one or more active pharmaceutical ingredients. The gastric residence article should be expelled from the stomach at the end of the desired residence time and be easily discharged from the patient. Currently, gastric residence articles are typically provided with linking components that undergo strength weakening processes in the gastric environment, such as degradation, dissolution, dissociation, or mechanical weakening, causing partial deployment arms to separate from the elastic central component or allowing the deployment arm to fold relative to the elastic central component within the gastrointestinal tract, ultimately enabling the expulsion of the elastic central component and deployment arm from the body.

To ensure the gastric residence article can reside in the gastric cavity for a sufficient duration, the deployment arm need to be provided with adequate mechanical strength to avoid structural damage due to gastric peristaltic pressure. The deployment arm are generally set to have a hardness of at least 50D. Therefore, during gastrointestinal transit, especially when the gastric residence article is compressed by the gastric wall and enters the smaller-volume small intestine stage, the rigid ends of the deployment arm can easily scratch or bruise the inner wall of the small intestine, leading to damage and inflammation of the small intestinal mucosa, thereby affecting the medical application of the gastric residence article.

### SUMMARY

Therefore, there is a need to provide a gastric residence article that solves the problem that after the gastric residence article is squeezed into the small intestine, the ends of the deployment arm easily scratch the mucosa of the inner wall of the small intestine, limiting the medical application of the gastric residence article.

To achieve the above objective, the present invention provides a gastric residence article, comprising:
an elastic central component;
a plurality of deployment arms, each deployment arm comprising a fixed end and a free end, the fixed end connected to the elastic central component, the plurality of deployment arms circumferentially fixed around the elastic central component, at least one of the deployment arms is loaded with an active substance;
an elastic end cap disposed at the free end of the deployment arm, the elastic end cap configured to undergo at least 20% elastic deformation in the direction of pressure when subjected to a pressure of 2 N,
the gastric residence article comprising a folded configuration and an expanded configuration, the elastic central component configured to provide an elastic force to transition the gastric residence article from the folded configuration to the expanded configuration.

Further, the elastic end cap has a Shore hardness of 20A to 80A.

For elastic end caps of the same structure, the lower the hardness, the lower the elastic modulus of the elastic end cap, and the greater the elastic deformation of the elastic end cap when subjected to gastrointestinal peristaltic pressure, thereby effectively avoiding hard collisions with the inner wall of the gastrointestinal tract and reducing inner wall injury.

Further, the elastic end cap has a Shore hardness of 20A to 60A. Experiments show that preferably the Shore hardness of the elastic end cap does not exceed 60A, which minimizes the probability of damage to the gastrointestinal tract by the gastric residence article. Meanwhile, a Shore hardness greater than 20A ensures a firm connection between the elastic end cap and the deployment arm.

Further, the deployment arm includes a first segment and a second segment in the axial direction, and the hardness of the first segment, the second segment, and the elastic end cap decreases in sequence. When the second segment of the deployment arm, which has a lower hardness, is subjected to gastrointestinal peristaltic force, it can also bend relative to the first segment, thereby further avoiding scratching of the intestinal inner wall.

Further, the elastic end cap has a hollow structure. The hollow structure provides the elastic end cap with a lower elastic modulus. For elastic end caps made of the same material, the hollow structure further enhances the deformation capability of the elastic end cap.

Further, the length of the deployment arm is at least 5 mm, and the ratio of the length of the elastic end cap to the length of the deployment arm is 0.08 to 0.5. The axial length of the elastic end cap cannot be too short; if too short, it cannot provide an elastic buffering effect. If too long, the gastric residence article cannot achieve a good gastric residence effect, and the elastic end cap is more likely to detach from the deployment arm.

Further, the elastic end cap comprises a first end surface and a second end surface, the first end surface being bonded to the free end of the deployment arm, and a curved or inclined transition being provided between the second end surface and the side surface.

Further, curved transitions are provided between the side surfaces of the deployment arm and between the side surfaces of the elastic end cap. Curved or inclined transitions can further avoid scratching of the gastric inner wall tissue by excessively sharp ends or edges.

Further, the deployment arm and the elastic end cap are thermally fused, wherein the thermal fusion is performed in one of the following manners:
(1) a molten adhesive is provided at the free end of the elastic end cap and/or the deployment arm, and the molten adhesive is heated and melted to bond the deployment arm and the elastic end cap;
(2) direct heating and thermal fusion bonding is performed between the free end of the elastic end cap and the free end of the deployment arm.

Further, a positioning structure is provided between the free end of the elastic end cap and the free end of the deployment arm. After the elastic end cap and the free end of the deployment arm are fixed via the positioning structure, the free ends are bonded together.

Further, the positioning structure is one of the following structures:
(1) the positioning structure comprises a protrusion and a groove respectively provided on the free end of the deployment arm and the elastic end cap, the protrusion and the groove engaging with each other, or vice versa;
(2) the positioning structure comprises an axial through hole provided in the elastic end cap and a melt material that enters the axial through hole during thermal fusion bonding and cools and solidifies therein.

Fixing the deployment arm and the elastic end cap via the positioning structure before performing heating and thermal fusion bonding can effectively improve the fixation effect between the elastic end cap and the deployment arm.

In manner (2), when the elastic end cap and the deployment arm are thermally fused, the melt material enters the axial through hole and cools to form the positioning structure. The melt material cooling in the axial through hole enhances the bonding and fixing effect, and the portion of the axial through hole not filled with the melt material constitutes the hollow structure.

On the other hand, the present invention provides a gastric residence article, comprising: an elastic central component;
a plurality of deployment arms, each deployment arm comprising a first rigid arm, a flexible segment, and a second rigid arm connected in sequence;
the first rigid arm connected to the elastic central component, the plurality of deployment arms circumferentially fixed on the elastic central component, at least one of the deployment arms is loaded with an active substance,
the flexible segment having a flexural modulus lower than that of the first rigid arm and the second rigid arm;
the gastric residence article comprising a folded configuration and an expanded configuration, the elastic central component providing an elastic force to transition the gastric residence article from the folded configuration to the expanded configuration.

Further, the flexural modulus of the flexible segment is less than 26.9 MPa.

Further, the elastic central component and the first rigid arm have an expanded diameter of at least 2 cm.

The above technical solutions have the following beneficial effects:
(1) The present invention provides a gastric residence article comprising an elastic end cap. In the gastrointestinal environment, especially when the gastric residence article is squeezed from the gastric cavity into the smaller-volume small intestine, since the free end of the deployment arm is terminated with the elastic end cap, under the force of gastrointestinal peristalsis, the elastic end cap will undergo at least 20% elastic deformation, thereby avoiding sharp collisions with the inner wall of the gastrointestinal tract and effectively preventing damage and inflammation to the gastrointestinal inner wall.
(2) On the other hand, the present invention discloses a gastric residence article comprising a deployment arm with a flexible segment. The flexible segment is configured to allow the two rigid arms connected thereby to bend relative to each other when subjected to pressure, providing a certain buffering effect when subjected to pressure from the gastrointestinal inner wall, thereby avoiding the end of the second rigid arm from scratching the gastrointestinal inner wall.

### BRIEF DESCRIPTION OF DRAWINGS

FIG. 1 is a schematic diagram showing the state of the gastric residence article in an expanded configuration within the gastric cavity according to a specific embodiment.

FIG. 2 is a schematic diagram showing the state of the gastric residence article about to be expelled from the gastric cavity according to a specific embodiment.

FIG. 3 is a schematic diagram showing the state of the gastric residence article in the duodenum according to a specific embodiment.

FIG. 4 is a structural diagram of the gastric residence article in an expanded configuration according to a specific embodiment.

FIG. 5 is a structural diagram of the gastric residence article in a folded configuration according to a specific embodiment.

FIG. 6 is an internal structural diagram of the elastic central component according to a specific embodiment.

FIG. 7 is an axial cross-sectional structural diagram of the gastric residence article according to a specific embodiment.

FIG. 8 is a schematic diagram of a testing method for elastic deformation of the elastic end cap according to a specific embodiment.

FIG. 9 is a schematic cross-sectional view of an elastic end cap comprising a hollow structure according to a specific embodiment.

FIG. 10 is a schematic cross-sectional view of one embodiment of the positioning structure according to a specific embodiment.

FIG. 11 is a schematic cross-sectional view of another embodiment of the positioning structure according to a specific embodiment.

FIG. 12 is a structural diagram of a gastric residence article according to another embodiment.

FIG. 13 is a structural diagram of a gastric residence article according to another embodiment.

FIG. 14 is a schematic diagram of a bending test for the second rigid arm according to a specific embodiment.

FIG. 15 is a schematic diagram of the bending angle of the second rigid arm according to a specific embodiment.

FIG. 16 is a sampling image of the duodenum of a test dog after animal testing using the gastric residence article of Example 1.

FIG. 17 is a sampling image of the duodenum of a test dog after animal testing using the gastric residence article of Comparative Example 2.

### DESCRIPTION OF REFERENCE NUMERALS

1: Elastic central component; 11: Elastic alloy member; 12: Polymer outer layer; 121: Central region; 122: Distal region;
2: Deployment arm; 21: First segment; 22: Second segment; 23: Second rigid arm; 24: First rigid arm; 25: Flexible segment;
3: Linking component;
4: Elastic end cap; 41: Curved transition; 42: Axial through hole;
5: Positioning structure; 51: Groove; 52: Protrusion; 53: Melt material;
10: Gastric residence article; 20: Gastric cavity; 30: Small intestine; 40: Workbench; 50: Indenter; 60: Sliding table; 70: Bending side stop.

### DETAILED DESCRIPTION OF THE EMBODIMENTS

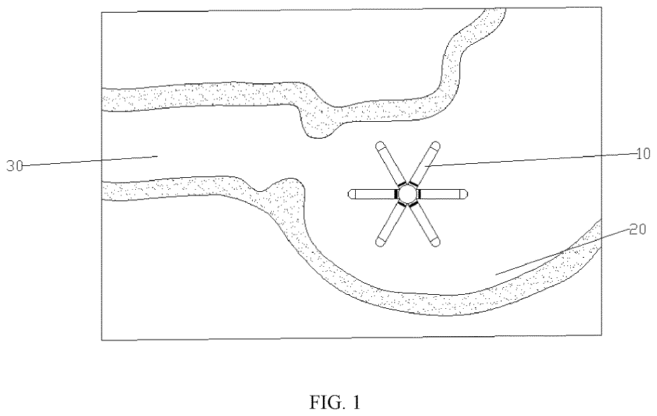

FIG. 1 shows the gastric residence article 10 in an expanded configuration within the gastric cavity 20. At this time, since the expanded diameter of the gastric residence article 10 is larger than the pylorus of the stomach, and the gastric residence article 10 has sufficient resistance to withstand gastric peristaltic pressure, it can reside in the gastric cavity 20 for a sufficient duration to release the active substance.

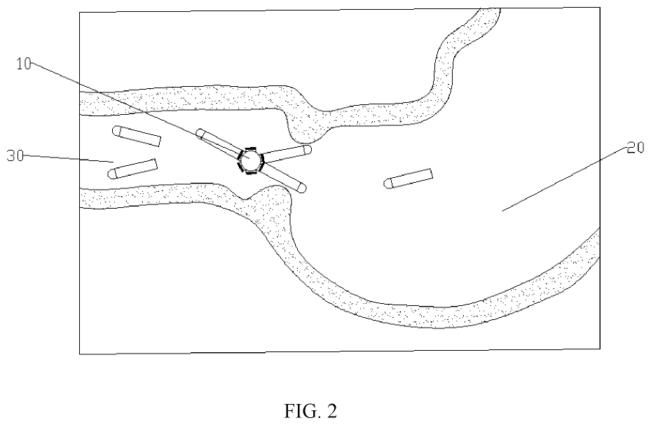

When the residence period expires, ideally all deployment arms separate simultaneously from the elastic central component, allowing expulsion from the gastric cavity 20 into the small intestine 30. However, due to the complexity of the environment in the gastric cavity 20, some deployment arms may detach prematurely. After losing some deployment arms, the gastric residence article 10 may be compressed under gastric peristaltic pressure and enter the small intestine 30. FIG. 2 schematically shows the gastric residence article 10 being squeezed through the pylorus into the duodenum of the small intestine 30 after some deployment arms break.

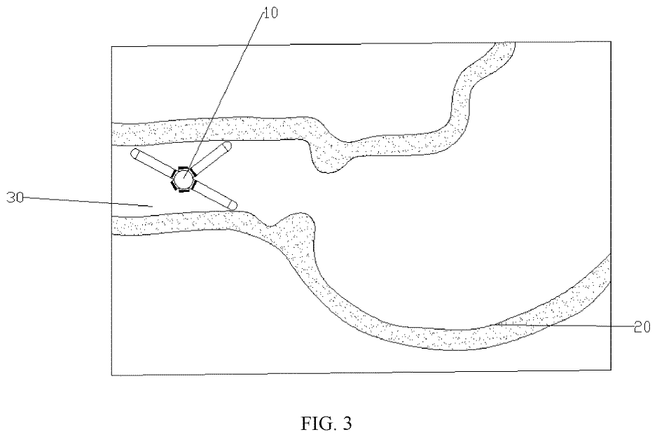

As shown in FIG. 3, after the gastric residence article 10 enters the small intestine 30, some deployment arms remain connected to the elastic central component, which still provides a certain compressive resistance to the deployment arm. Due to the smaller lumen of the small intestine 30, when the peristaltic pressure of the small intestine 30 compresses and transports the gastric residence article 10, the relatively hard ends of the deployment arm can easily scratch the interior of the small intestine 30.

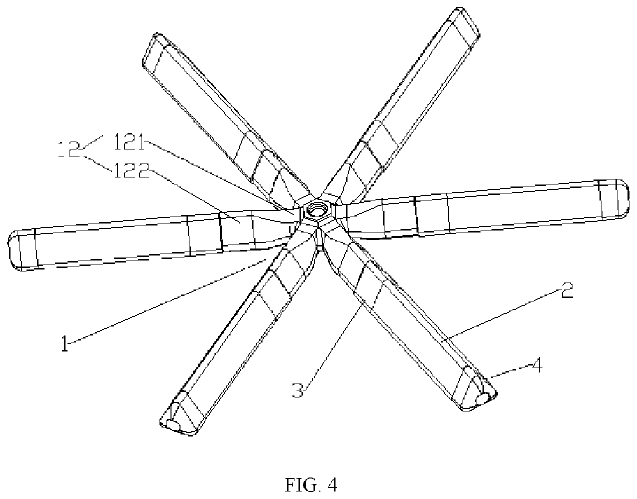

Referring to FIGS. 4-7, one aspect of the present invention provides a gastric residence article comprising an elastic central component 1, a plurality of deployment arms 2, a plurality of linking components 3, and a plurality of elastic end caps 4. The two ends of the deployment arm 2 correspond one-to-one to the linking component 3 and the elastic end cap 4, respectively. The deployment arm 2 is connected to the elastic central component 1 via the linking component 3.

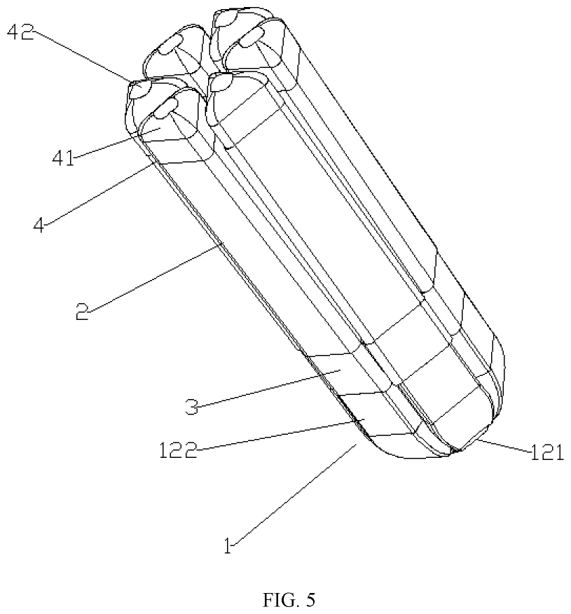

FIG. 4 shows the gastric residence article in an expanded configuration, and FIG. 5 shows it in a folded configuration. In the folded configuration, the deployment arm 2 of the gastric residence article close together tightly, allowing the folded configuration to be filled into a capsule shell. After the capsule is swallowed into the body, the capsule shell dissolves in the stomach, and the elastic center provides a restoring force to deploy the deployment arm 2 of the gastric residence article into the expanded configuration.

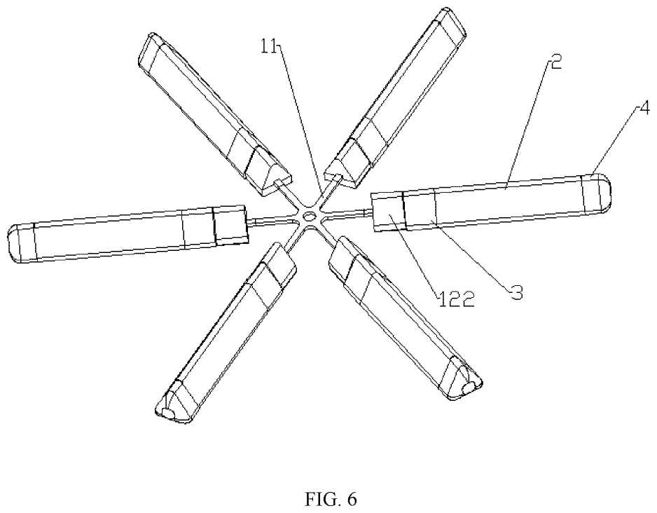

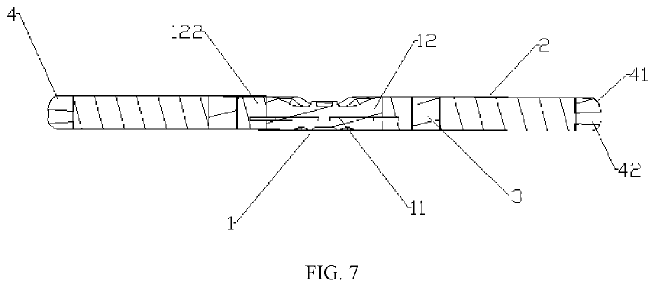

FIGS. 6-7 show the internal structure of the elastic central component 1 of the gastric residence article. In the gastric cavity, the elastic central component 1 provides sufficient resistance to the gastric residence article to resist gastric peristaltic pressure and prevent the expanded configuration from refolding. In this embodiment, the elastic central component 1 comprises an elastic alloy member 11 fixed within a polymer outer layer 12. The elastic alloy member 11 provides the main resistance.

The elastic alloy member 11 may be an elastic alloy or a memory alloy, such as nickel-titanium alloy, which has sufficient biosafety. In this embodiment, the elastic alloy comprises an integrally formed central metal ring and six metal ribs extending circumferentially, the metal ribs being connected to one end of the deployment arm 2 via the linking components 3, respectively.

The polymer outer layer 12 is selected from one or more of polypropylene, polystyrene, polyvinyl chloride, synthetic rubber, phenolic resin, neoprene, nylon, polyacrylonitrile, PVB, silicone, acrylonitrile butadiene styrene, high-density polyethylene, polycarbonate, nylon, acrylic, polyethylene terephthalate, polybutylene terephthalate, acetal, polyimide, polyurethane, and epoxy resin.

As shown in FIGS. 4-7, the polymer outer layer 12 comprises a central region 121 with a lower hardness and a distal region 122 with a higher hardness. The metal ribs are inserted into the distal region 122, which is connected to the linking component 3.

In another embodiment, the elastic central component 1 is entirely made of an elastic polymer material.

In another embodiment, the elastic alloy component consists of the elastic alloy member 11.

To conveniently control the residence time of the gastric residence article, a linking component 3 is provided between the elastic central component 1 and the deployment arm 2. The linking component 3 is configured to cause separation of the deployment arm 2 from the elastic central component 1 after strength weakening. The strength weakening process may include degradation, dissolution, dissociation, or mechanical weakening, resulting in the loss of the expanded configuration shape of the gastric residence article.

In one embodiment, the linking component 3 may be dissolved by mixing a water-soluble material into its matrix.

The plurality of deployment arms 2 are circumferentially fixed around the elastic central component 1, and at least one deployment arm 2 is loaded an active substance; the active substance may be a therapeutic agent or a diagnostic agent.

The gastric residence article comprises at least three deployment arms 2. In this embodiment, the gastric residence article has six deployment arms 2.

The deployment arm 2 is made of at least one material selected from polypropylene, polystyrene, polyvinyl chloride, synthetic rubber, phenolic resin, neoprene, nylon, polyacrylonitrile, PVB, silicone, acrylonitrile butadiene styrene, high-density polyethylene, polycarbonate, acrylic, polyethylene terephthalate, polybutylene terephthalate, acetal, polyimide, polyurethane, and epoxy resin.

The deployment arm 2 has a fixed end and a free end. The fixed end of the deployment arm 2 is connected to the distal region 122 of the elastic central component 1 via the linking component 3, and the plurality of deployment arms 2 are circumferentially fixed on the elastic central component 1.

An elastic end cap 4 is provided at the free end of the deployment arm 2. To avoid scratching the inner wall of the gastrointestinal tract, the elastic end cap 4 is configured to undergo at least 20% elastic deformation in the direction of pressure when subjected to a pressure of 2 N.

In some embodiments, when subjected to a pressure of 2 N, the elastic deformation of the elastic end cap 4 is 20%-25%, 25%-30%, 30%-35%, 35%-40%, 40%-50%, or 20%-50%.

To enable the gastric residence article to reside in the gastric cavity, the expanded diameter of the gastric residence article in the expanded configuration needs to be at least 2 cm or more.

“Expanded diameter” is defined as: when the gastric residence article is in the expanded configuration, connecting the tips of the respective elastic end caps 4 to form a polygon, and the diameter of the circumscribed circle of this polygon.

In some embodiments, the diameter of the elastic central component 1 is 10 mm, the length of a single deployment arm 2 is 5-20 mm, and the length of the elastic end cap 4 is 1.6 mm-5 mm.

In some embodiments, the ratio of the length of the elastic end cap 4 to the length of the deployment arm 2 is 0.08-0.5. The axial thickness, i.e., the length, of the elastic end cap 4 cannot be too short; if too short, it cannot provide an elastic buffering effect. If too long, the gastric residence article cannot achieve a good gastric residence effect, and the elastic end cap 4 is more likely to detach from the deployment arm 2.

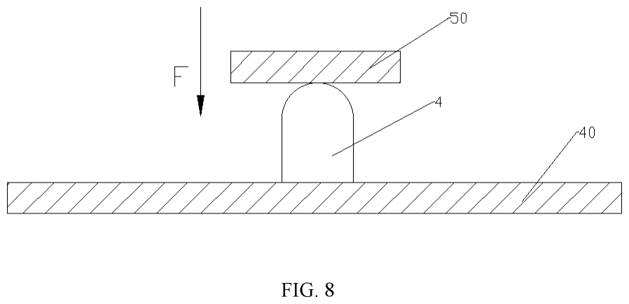

As shown in FIG. 8, a testing method for the deformation of the elastic end cap 4 is displayed. Using a tensile tester, the elastic end cap 4 is fixed on the workbench 40 of the tensile tester. Initially, the bottom surface of the indenter 50 of the tensile tester abuts the top of the elastic end cap 4, with no pressure applied to the elastic end cap 4 at this time. After setting the program, the indenter 50 is controlled to press down. When the force sensor on the indenter 50 reaches the specified force value of 2 N, the distance traveled by the motor is the deformation amount of the elastic end cap 4.

The elastic end cap 4 can achieve the deformation performance parameters within the corresponding range through various means. In the present invention, the elastic end cap 4 meets the elastic deformation performance requirements through material selection and/or the provision of a hollow structure.

Regarding material selection, the elastic end cap 4 is made of a material with a Shore hardness not higher than 80A and not lower than 20A. For elastic end caps 4 of the same structure, the lower the hardness, the lower the elastic modulus of the elastic end cap 4, and the greater the elastic deformation when subjected to gastrointestinal peristaltic pressure, i.e., the higher the relative elastic deformation, thereby providing a good buffering effect, effectively avoiding hard collisions with the inner wall of the gastrointestinal tract, and reducing inner wall injury.

In some embodiments, the deployment arm 2 has a Shore hardness of at least 50D, and the elastic end cap 4 has a Shore hardness of 20A-60A. The deployment arm 2 is at least 50D to avoid bending under pressure. Experiments show that selecting the Shore hardness of the elastic end cap 4 to be no more than 60A minimizes the probability of damage to the gastrointestinal tract by the gastric residence article. Meanwhile, a Shore hardness greater than 20A ensures a firm connection between the elastic end cap 4 and the deployment arm 2.

In some specific embodiments, the elastic end cap 4 is made of materials with Shore hardnesses of 20A, 30A, 40A, 50A, 60A, 70A, and 80A.

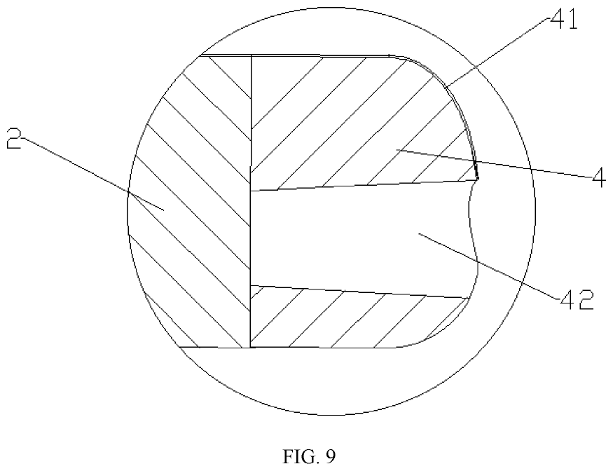

As shown in FIG. 9, regarding structural arrangement, a hollow structure is provided in the elastic end cap 4. For elastic end caps 4 made of the same material, the hollow structure can further enhance the deformation capability of the elastic end cap 4.

In FIG. 9, the hollow structure is an axial through hole 42.

In some embodiments, the elastic end cap 4 may also be provided with a hollow cavity internally to achieve the hollow structure.

In some embodiments, the elastic end cap 4 is made of one or more polymers selected from polyurethane, polyether-polyamide copolymer, thermoplastic elastomer, thermoplastic polyurethane, polycaprolactone-polylactic acid copolymer, poly(trimethylene carbonate), poly(glycerol sebacate), natural rubber, and silicone.

The elastic end cap 4 comprises, along its axial direction, a first end surface and a second end surface opposite thereto. The first end surface is bonded to the free end of the deployment arm 2, and a curved transition 41 or an inclined transition is provided between the second end surface and the side surface of the elastic end cap 4; and/or curved transitions 41 are provided between the side surfaces of the deployment arm 2 and between the side surfaces of the elastic end cap 4.

Curved or inclined transitions can further avoid excessively sharp ends from scratching the gastric inner wall tissue.

The curved transition 41 mentioned in the present invention can be implemented in the following ways:
Method 1: The curved transition 41 is an arc surface transition.
Method 2: The curved transition 41 is a streamlined transition.
Method 3: The curved transition 41 is an elliptical surface transition.
Method 4: The curved surface at the curved transition 41 is not limited to a single curved surface; that is, the curvature of the curved surface may vary and may be formed by splicing multiple curved surfaces (which may also be regarded as arc surfaces).

In this embodiment, the curved transition 41 is exemplified by an arc surface transition.

In some embodiments, the deployment arm 2 and the elastic end cap 4 both have a triangular cross-sectional shape, and the junctions between the side surfaces of the deployment arm 2 and the junctions between the side surfaces of the elastic end cap 4 are all curved transitions 41.

The edges of the deployment arm 2 and the elastic end cap 4 are also provided with curved transitions 41 to avoid excessively sharp edges scratching the gastric inner wall tissue.

The deployment arm 2 and the elastic end cap 4 can be bonded via an adhesive or by thermal fusion bonding. The thermal fusion bonding is performed in one of the following manners:
(1) a molten adhesive is provided on the first end surface of the elastic end cap 4 and/or the free end of the deployment arm 2, and the molten adhesive is heated and melted to bond the deployment arm 2 and the elastic end cap 4;
(2) direct heating and thermal fusion bonding is performed between the first end surface of the elastic end cap 4 and the free end of the deployment arm 2; when the material of the deployment arm 2 and/or the elastic end cap 4 itself is a hot-melt material, light heating can be directly applied at the joint for thermal fusion bonding.

A positioning structure 5 is provided between the first end surface of the elastic end cap 4 and the free end of the deployment arm 2. After the first end surface of the elastic end cap 4 and the free end of the deployment arm 2 are fixed via the positioning structure 5, thermal fusion bonding is performed between the first end surface of the elastic end cap 4 and the free end of the deployment arm 2. The positioning mechanism strengthens the connection between the elastic end cap 4 and the deployment arm 2.

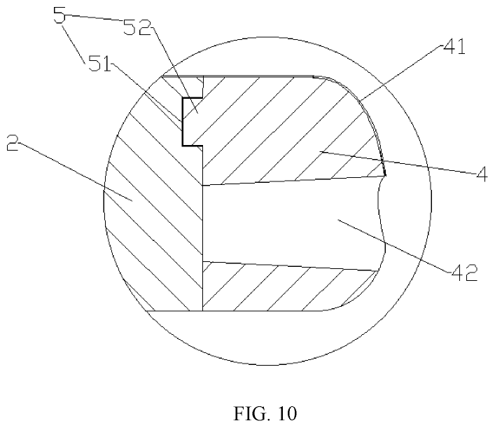

As shown in FIG. 10, the positioning structure 5 comprises a protrusion 52 and a groove 51 provided on the free end of the deployment arm 2 and the elastic end cap 4, respectively. The protrusion 52 and the groove 51 engage with each other, or vice versa. The elastic end cap 4 and the deployment arm 2 are first positioned and fixed via the interlocking nested structure, followed by laser heating for thermal fusion bonding, effectively strengthening the fixation effect between the elastic end cap 4 and the deployment arm 2.

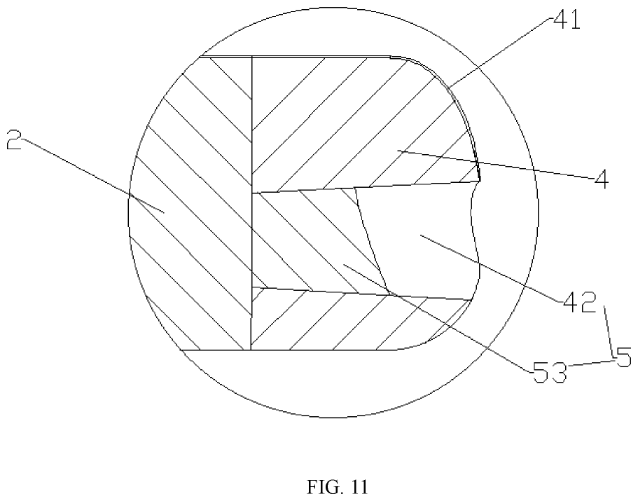

As shown in FIG. 11, the positioning structure 5 comprises an axial through hole 42 provided in the elastic end cap 4 and a melt material 53 that enters the axial through hole 42 during thermal fusion bonding and cools and solidifies therein. When the elastic end cap 4 and the deployment arm 2 are thermally fused, the melt material 53 enters the axial through hole 42, cools, and solidifies to form the positioning structure 5.

In the embodiment of FIG. 11, the heated molten melt material 53 flows into the axial through hole 42 and cools to bond the elastic end cap 4 and the deployment arm 2. Meanwhile, after the melt material 53 cools in the axial through hole 42, the unfilled portion of the axial through hole 42 constitutes the hollow structure, providing deformation space for the elastic end cap 4.

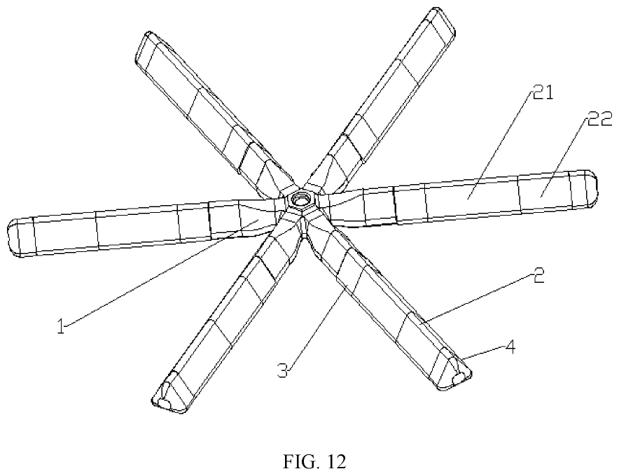

As shown in FIG. 12, to enable the deployment arm 2 to bend relatively and further avoid scratching the gastric inner wall, the deployment arm 2 comprises a first segment 21 and a second segment 22 along the axial direction, with the hardness of the first segment 21, the second segment 22, and the elastic end cap 4 decreasing sequentially. When the second segment 22 of the deployment arm 2, which has a lower hardness, is subjected to gastrointestinal peristaltic force, it can also bend relative to the first segment 21, thereby further avoiding scratching of the intestinal inner wall.

In some embodiments, the first segment 21 is made of Lubrizol TPU: TT-2075D-B40, and the second segment 22 is made of Covestro TPU: 1350D.

In the present invention, the elastic end cap 4 can be configured to be non-drug-loaded, and material selection can be used to make the diffusion rate of the active substance on the elastic end cap 4 lower than that on the deployment arm 2.

When the elastic end cap 4 is squeezed by the gastric inner wall, since the elastic end cap 4 does not contain active drugs, it does not affect the drug release profile of the gastric residence article in the gastric environment. The elastic end cap 4 seals the free end surface of the deployment arm 2, so that the active substance can only be released from the side, prolonging the release time of the active drug within the deployment arm 2. Simultaneously, the factor of pressure-induced release from the free end surface is eliminated, allowing for more effective control of the active drug release profile.

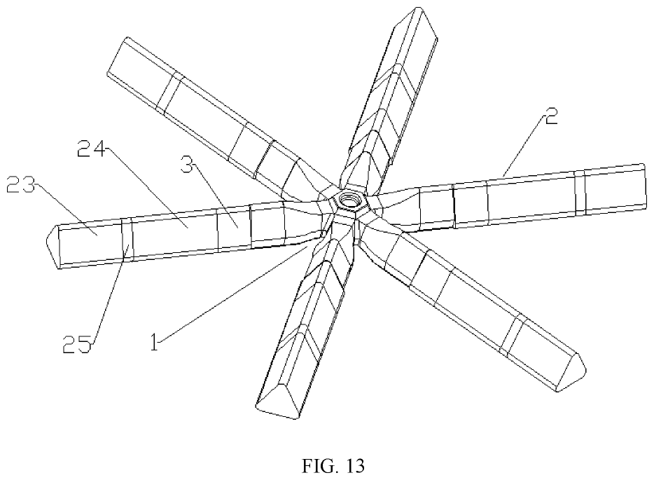

Referring to FIG. 13, another aspect of the present invention provides a gastric residence article comprising an elastic central component 1, a linking component 3, and a plurality of deployment arms 2. The gastric residence article comprises a folded configuration and an expanded configuration, and the elastic central component 1 provides an elastic force to transition the gastric residence article from the folded configuration to the expanded configuration.

The deployment arm 2 comprises a first rigid arm 24, a flexible segment 25, and a second rigid arm 23 connected in sequence. The flexible segment 25 has a rigidity lower than that of the first rigid arm 24. The first rigid arm 24 is connected to the elastic central component 1, the plurality of deployment arms 2 are circumferentially fixed around the elastic central component 1, and at least one deployment arm 2 is loaded an active substance. The flexible segment 25 has a flexural modulus lower than that of the first rigid arm 24 and the second rigid arm 23, allowing the first rigid arm 24 and the second rigid arm 23 to bend relative to each other when gastrointestinal peristalsis applies a peristaltic force to the gastric residence article within the gastrointestinal tract.

In some embodiments, the flexural modulus of the flexible segment 25 does not exceed 26.9 MPa, thereby allowing the first rigid arm 24 and the second rigid arm 23 to bend relative to each other by a certain angle.

In some embodiments, the material of the flexible segment 25 can be selected from materials with hardnesses such as TPU-50A, TPU-60A, TPU-70A, and TPU-85A, wherein TPU-85A has a flexural modulus of 26.9 MPa and TPU-70A has a flexural modulus of 14.5 MPa.

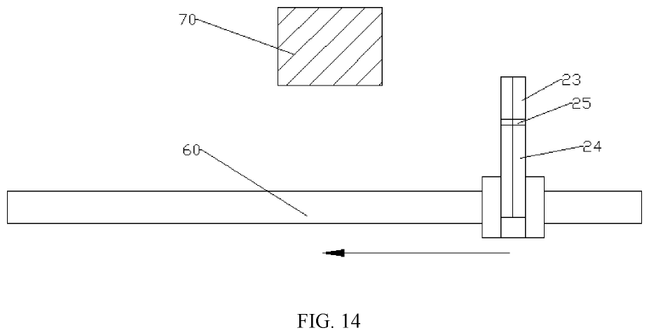

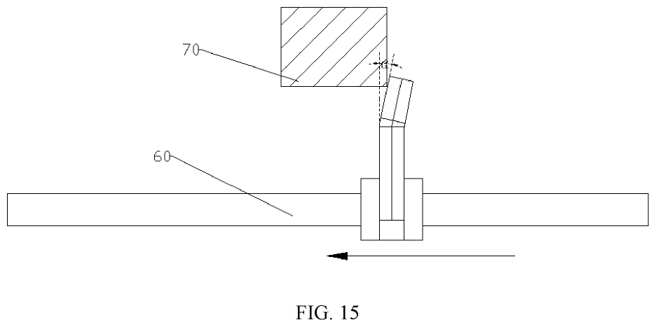

The bending test results of the deployment arm 2 are shown in FIGS. 14-15. The setup includes a sliding table 60 and a bending side stop 70. The first rigid arm 24 of the deployment arm 2 of the gastric residence article is fixed on the sliding table 60. The bending side stop 70 is equipped with a pressure sensor. The sliding table 60 drives the deployment arm 2 to slide, and the bending side stop 70 abuts the end of the second rigid arm 23, causing the second rigid arm 23 to bend relative to the first rigid arm 24. In FIG. 15, angle α is the bending angle of the second rigid arm 23.

In some embodiments, the flexible segment 25 enables the second rigid arm 23 to bend at least 5° relative to the first rigid arm 24 when a pressure of 2 N is applied to the end segment of the second rigid arm 23.

To avoid premature expulsion of the gastric residence article from the stomach, the elastic central component 1 and the first rigid arm 24 have an expanded diameter of at least 2 cm.

The ratio of the length of the first rigid arm 24 to the length of the second rigid arm 23 is 2:1 to 8:2. In specific embodiments, the length ratio of the first rigid arm 24 to the second rigid arm 23 may be 2:1, 3:1, or 4:1.

To detail the technical content, structural features, achieved objectives, and effects of the technical solutions, the following describes specific embodiments in conjunction with the accompanying drawings.

### Examples 1-5

Preparation of the gastric residence article shown in FIG. 4, comprising the following process:
1. Using nickel-titanium alloy as the elastic alloy member 11, and injection molding a thermoplastic polyurethane elastomer externally as the polymer outer layer 12, with the central region 121 being Lubrizol TPU: PC-3575A and the distal region 122 being Covestro TPU: 1350D, thereby forming the elastic central component 1;
2. Mixing polycaprolactone (PCL) and hydroxymethyl cellulose (HPMC-AS-HG), extruding the mixture using an extruder, and then cutting it into 3 mm segments to form the linking component 3;
3. Mixing polycaprolactone (PCL) powder (50D), polyolefin powder, active ingredients, and auxiliary materials such as silica, extruding the mixture through an extruder, and cutting it into 10 mm segments to form the deployment arm 2;
4. Using silicone-20A, TPU-40A, TPU-60A, TPU-70A, and TPU-80A materials respectively, extruding them through an extruder to form shapes, and cutting them to form the elastic end cap 4;
5. Sequentially laser heating and thermally fusing the elastic central component 1, the linking component 3, the deployment arm 2, and the elastic end cap 4 together.

### Example 6

Using TPU-60A material, extruding it through an extruder, cutting it to form an elastic end cap, axially drilling it to form an axial through hole, and thermally fusing it with the deployment arm to prepare the gastric residence article.

### Comparative Example 1

Using silicone-10A material to prepare the elastic end cap and bonding it to the free end of the deployment arm. Since the elastic end cap made of silicone-10A material is too soft, it easily detaches from the deployment arm.

### Comparative Example 2

Using TPU-90A material to prepare the elastic end cap and thermally fusing it with the deployment arm to prepare the gastric residence article.

### Example 7

The elastic end caps prepared in Examples 1-6 and Comparative Example 2 were tested for elastic deformation under a pressure of 2 N. The results are shown in Table 1 below:

TABLE 1
Test Results of Elastic Deformation of Elastic End Caps

| | Axial Length | Indenter | Relative |
|---|---|---|---|
| | of Elastic | Displacement | Deformation |
| | End Cap (mm) | (mm) | (%) |
| Example 1 | 1.817 | 0.858 | 47.22 |
| Example 2 | 1.784 | 0.693 | 38.85 |
| Example 3 | 1.913 | 0.635 | 33.19 |
| Example 4 | 1.925 | 0.501 | 26.02 |
| Example 5 | 1.862 | 0.384 | 20.62 |
| Example 6 | 1.836 | 0.687 | 37.42 |
| Comparative | 1.865 | 0.326 | 17.48 |
| Example 2 | | | |

### Example 8, Different Animal Tests

The gastric residence articles were filled into corresponding capsule shells and fed to test dogs separately. The activity of the test dogs was observed. X-rays showed that the gastric residence articles resided in the digestive tract of the test dogs for about 7-14 days. The dogs were fed once every 7 days, and the gastric residence article was fed three times consecutively. The state of the test dogs was observed. The test results are shown in Table 2 below.

TABLE 2
Animal Tests of Gastric Retention Devices

| | Relative | Hollow | Condition of Test Dogs |
|---|---|---|---|
| Example | Deformation (%) | Structure | During Administration |
| 1 | 47.22 | No | No obvious symptoms |
| 2 | 38.85 | No | No obvious symptoms |
| 3 | 33.19 | No | No obvious symptoms |
| 4 | 26.02 | No | Slight decrease in |
| | | | food intake after |
| | | | the third feeding |
| 5 | 20.62 | No | Slight decrease in |
| | | | food intake after |
| | | | the third feeding |
| 6 | 37.42 | Yes | No obvious symptoms |
| Comparative | 17.48 | No | Reduced food intake |
| Example 2 | | | after the second |
| | | | feeding; slight fever |
| | | | accompanied by |
| | | | hematochezia after |
| | | | the third feeding |

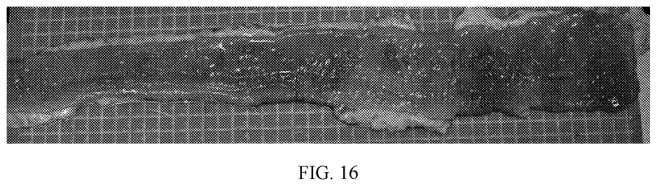

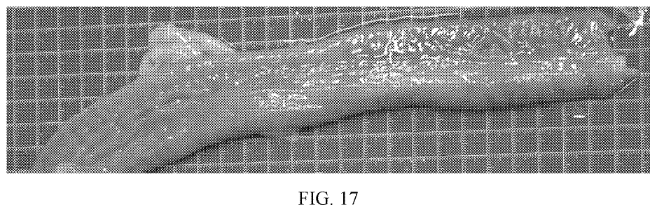

The test dogs from Example 6 and Comparative Example 2 were dissected, and samples were taken from the duodenum of the small intestine. As shown in FIGS. 16 and 17, no changes or trauma signs were found in the sample in FIG. 16, indicating that the treatment was safe. In the sample in FIG. 17, the inner wall of the duodenum had large sections of congestion (purple inner wall color) and partial tissue depression, presumably caused by scratching/bruising from the ends of the deployment arms.

### Example 9

Preparation of the gastric residence article shown in FIG. 13, comprising the following process:
1. Using nickel-titanium alloy as the elastic alloy member, and injection molding a thermoplastic polyurethane elastomer externally as the polymer outer layer, with the central region being Lubrizol TPU: PC-3575A and the distal region being Covestro TPU: 1350D, thereby forming the elastic central component 1;
2. Mixing polycaprolactone (PCL) and hydroxymethyl cellulose (HPMC-AS-HG), extruding the mixture using an extruder, and then cutting it into 3 mm segments to form the linking component 3;
3. Mixing polycaprolactone (PCL) powder, polyolefin powder, active ingredients, and auxiliary materials such as silica, extruding the mixture through an extruder, and cutting it into 8 mm and 4 mm segments to form the first rigid arm 24 and the second rigid arm 23;
4. Using a material with a hardness of TPU-70A (flexural modulus 14.5 MPa), extruding it through an extruder, and cutting it to form the flexible segment 25;
5. Sequentially laser heating and thermally fusing the first rigid arm 24, the flexible segment 25, and the second rigid arm 23 to form the deployment arm;
6. Sequentially laser heating and thermally fusing the elastic central component 1, the linking component 3, and the deployment arm 2.

In the present invention, by providing an elastic end cap capable of undergoing at least 20% elastic deformation at the free end of the deployment arm and/or configuring the deployment arm as two rigid arms capable of relative bending, when the gastric residence article enters the small intestine and the small intestine peristalsis applies pressure to the deployment arm, the elastic end cap undergoes elastic deformation or the second rigid arm bends. This provides a certain buffering effect, increases the contact area, avoids sharp collisions, and thereby reduces the possibility of damage to the small intestinal mucosa.

It should be noted that relational terms such as “first” and “second” are used herein solely to distinguish one entity or operation from another entity or operation, and do not necessarily require or imply any actual relationship or order between such entities or operations. Moreover, the terms “comprises,” “comprising,” or any other variation thereof are intended to cover non-exclusive inclusion, such that a process, method, article, or terminal device that comprises a list of elements does not include only those elements but also includes other elements not expressly listed, or elements inherent to such process, method, article, or terminal device. Without further limitation, an element defined by the statement “comprises . . . ” or “including . . . ” does not exclude the presence of additional identical elements in the process, method, article, or terminal device that comprises the element. Furthermore, in this context, “greater than,” “less than,” “exceeds,” etc., are understood as excluding the stated number; “or more,” “or less,” “within,” etc., are understood as including the stated number.

Although the above embodiments have been described, those skilled in the art will appreciate that once the basic inventive concept is known, further changes and modifications may be made to these embodiments. Therefore, the above description is merely exemplary embodiments of the present invention and is not intended to limit the scope of patent protection of the present invention. Any equivalent structure or equivalent process transformation made using the contents of the description and drawings of the present invention, or any direct or indirect application thereof in other related technical fields, shall fall within the scope of patent protection of the present invention.

What is claimed is:

1. A gastric residence article, comprising:
an elastic central component;
a plurality of deployment arms, each deployment arm comprising a fixed end and a free end, the fixed end connected to the elastic central component, the plurality of deployment arms circumferentially fixed around the elastic central component, at least one of the deployment arms is loaded with an active substance;
an elastic end cap disposed at the free end of the deployment arm, the elastic end cap configured to undergo at least 20% elastic deformation in the direction of pressure when subjected to a pressure of 2 N;
the gastric residence article comprising a folded configuration and an expanded configuration, the elastic central component configured to provide an elastic force to transition the gastric residence article from the folded configuration to the expanded configuration.

2. The gastric residence article as claimed in claim 1, wherein the elastic end cap has a Shore hardness of 20A to 80A.

3. The gastric residence article as claimed in claim 2, wherein the elastic end cap has a Shore hardness of 20A to 60A.

4. The gastric residence article as claimed in claim 2, wherein the deployment arm includes a first segment and a second segment in the axial direction, and the hardness of the first segment, the second segment, and the elastic end cap decreases in sequence.

5. The gastric residence article as claimed in claim 1, wherein the elastic end cap has a hollow structure.

6. The gastric residence article as claimed in claim 1, wherein the length of the deployment arm is at least 5 mm, and the ratio of the length of the elastic end cap to the length of the deployment arm is 0.08-0.5.

7. The gastric residence article as claimed in claim 1, wherein the elastic end cap comprises a first end surface and a second end surface, the first end surface being bonded to the free end of the deployment arm, and a curved or inclined transition being provided between the second end surface and the side surface.

8. The gastric residence article as claimed in claim 7, wherein curved transitions are provided between the side surfaces of the deployment arm and between the side surfaces of the elastic end cap.

9. The gastric residence article as claimed in claim 1, wherein the deployment arm and the elastic end cap are thermally fused, wherein the thermal fusion is performed in one of the following manners:
(1) a molten adhesive is provided at the free end of the elastic end cap and/or the deployment arm, and the molten adhesive is heated and melted to bond the deployment arm and the elastic end cap;
(2) direct heating and thermal fusion bonding is performed between the free end of the elastic end cap and the free end of the deployment arm.

10. The gastric residence article as claimed in claim 9, wherein a positioning structure is provided between the free end of the elastic end cap and the free end of the deployment arm, and after the elastic end cap and the free end of the deployment arm are fixed via the positioning structure, the free ends are bonded together.

11. The gastric residence article as claimed in claim 10, wherein the positioning structure is one of the following structures:
(1) the positioning structure comprises a protrusion and a groove respectively provided on the free end of the deployment arm and the elastic end cap, the protrusion and the groove engaging with each other, or vice versa;
(2) the positioning structure comprises an axial through hole provided in the elastic end cap and a melt material that enters the axial through hole during thermal fusion bonding and cools and solidifies therein.

12. A gastric residence article, comprising:
an elastic central component;
a plurality of deployment arms, each deployment arm comprising a first rigid arm, a flexible segment, and a second rigid arm connected in sequence;
the first rigid arm connected to the elastic central component, the plurality of deployment arms circumferentially fixed on the elastic central component, at least one of the deployment arms is loaded with an active substance; the flexible segment having a flexural modulus lower than that of the first rigid arm and the second rigid arm;
the gastric residence article comprising a folded configuration and an expanded configuration, the elastic central component providing an elastic force to transition the gastric residence article from the folded configuration to the expanded configuration.

13. The gastric residence article as claimed in claim 12, wherein the flexural modulus of the flexible segment is less than 26.9 MPa.

14. The gastric residence article as claimed in claim 12, wherein the elastic central component and the first rigid arm have an expanded diameter of at least 2 cm.
